(19) United States
(12) Patent Application Publication
(10) Pub. No.: US 2024/0358770 A1
(43) Pub. Date: Oct. 31, 2024

(54) METHODS OF TREATING PODODERMATITIS

(71) Applicant: MICROBIAL DISCOVERY GROUP, LLC, Franklin, WI (US)

(72) Inventors: Elizabeth GALBRAITH, Wauwatosa, WI (US); Sona SON, Franklin, WI (US); Michael R. KING, Oak Creek, WI (US)

(21) Appl. No.: 18/293,189

(22) PCT Filed: Jul. 28, 2022
(86) PCT No.: PCT/US2022/038647

§ 371 Date: Jan. 29, 2024

Related U.S. Application Data
(60) Provisional application No. 63/227,069, filed on Jul. 29, 2021.

(51) Int. Cl. A61K 35/74; A23K 10/16; A61P 31/04; C12N 1/20; C12R 1/07

(52) U.S. Cl. CPC A61K 35/74; A23K 10/16; A61P 31/04; C12N 1/205; C12R 2001/07

(57) ABSTRACT

The invention relates to direct-fed microbials for use in Enterococcus species inhibition in animals. More particularly, the invention relates to isolated Bacillus strains A12, 681, 101, 235, 177, 86, and 300, and strains having all of the identifying characteristics of these strains, for a use comprising the above-mentioned use. The invention also relates to the use of isolated Bacillus strains A12, 681, 101, 235, 177, 86, and 300, and strains having all of the identifying characteristics of these strains, to prevent or reduce or control pododermatitis (footpad disease) in an animal.

### CROSS-REFERENCE TO RELATED APPLICATIONS

This application claims the benefit under 35 USC § 119(e) of U.S. Provisional Application Ser. No. 63/227,069, filed on Jul. 29, 2021, the entire disclosure of which is incorporated herein by reference.

### FIELD OF THE DISCLOSURE

The invention relates to direct-fed microbials for use in Enterococcus species inhibition in animals. More particularly, the invention relates to isolated Bacillus strains A12, 681, 101, 235, 177, 86, and 300, and strains having all of the identifying characteristics of these strains, for a use comprising the above-mentioned use. The invention also relates to the use of isolated Bacillus strains A12, 681, 101, 235, 177, 86, and 300, and strains having all of the identifying characteristics of these strains, to prevent or reduce or control pododermatitis (footpad disease) in an animal.

### BACKGROUND AND SUMMARY OF THE INVENTION

The development of pododermatitis or necrotic lesions on the paws of animals impacts the welfare of the animals and can affect animal productivity and the quality of animal meat, causing important economic losses. Furthermore, since animal paws are an important commodity in the international market due to popularity as a traditional delicacy, the presence of pododermatitis prevents exportation of affected animals, since importing countries do not accept animals with injured feet. Pododermatitis is thought to be caused by a combination of factors including poor water drinker design or management; diet composition; house temperature and humidity levels; litter type, quality, and quantity; and gut health. High levels of moisture in the litter are also thought to be of crucial importance, and recent evidence of bacterial involvement in pododermatitis has been demonstrated. In poultry, paw lesions have been correlated to systemic infection with Gram-positive cocci, specifically Enterococcus and Staphylococcus species. Though several measures have been reported to provide some effect in preventing or treating footpad lesions (enzymes, biotin, zinc and methionine), the lack of full effectiveness of these measures has led the animal industry to look for alternatives.

Enterococcus are gram-positive bacteria that commonly inhabit the gastrointestinal tracts of animals, such as poultry. Transmission of Enterococcus is via the oral and/or aerosol routes as well as from skin wounds, and naturally high levels of antibiotic resistance are found for E. faecalis and contribute to its pathogenicity. In broiler breeders, paw lesions have been correlated to systemic infection with gram-positive cocci, including Enterococcus faecalis. E. faecalis has also been specifically associated with endocarditis in broilers while other Enterococci have been linked to arthritis, osteomyelitis, spondylitis, and femoral head necrosis in broiler and broiler breeder flocks. Reducing Enterococcus quantities in the gastrointestinal tract or litter is a starting point for reducing transmission. The inventors have demonstrated that specific Bacillus strains A12 (NRRL No. B-67516), 681 (NRRL No. B-67515), 101 (NRRL No. B-67218), 235 (NRRL No. B-67219), 177 (NRRL No. B-67275), 86 (NRRL No. B-50944), and 300 (NRRL No. B-50943) can inhibit the growth of Enterococcus faecalis isolates from litter, such as poultry litter.

Applicant has developed direct-fed microbials that result in Enterococcus inhibition. The direct-fed microbials and compositions comprising the direct-fed microbials described herein offer a commercial benefit by providing Enterococcus inhibition in animals, such as agricultural animals. In addition, the direct-fed microbial compositions described herein result in a reduction or elimination in the use of antibiotics which reduces the overall cost of animal feed and reduces the development of antibiotic resistance. Applicant has also developed direct-fed microbials for reducing or preventing pododermatitis in animals, such as agricultural animals.

Methods and compositions are provided for inhibiting Enterococcus in animals. In various embodiments, the animal can be selected from the group consisting of a poultry species, a porcine species, a caprine species, a bovine species, an ovine species, an equine species, and a companion animal. In the embodiment where the animal is a poultry species, the poultry species can be selected from the group consisting of a broiler, a chicken, a layer, a breeder, a turkey, a turkey poult, a gosling, a duckling, a guineakeet, a pullet, a hen, a rooster, a cockerel, and a capon. In the embodiment where the animal is a porcine species, the porcine species can be selected from the group consisting of a grow finish pig, a nursery pig, a sow, and a breeding stock pig.

In various embodiments, the compositions for use in the methods described herein can be a commercial package, a feed additive for an animal feed composition, an additive for the drinking water of an animal, or an animal feed composition (e.g., a complete feed), each comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B-67516), a strain having all of the identifying characteristics of Bacillus strain 681 (NRRL No. B-67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, for inhibiting Enterococcus and/or preventing or reducing pododermatitis in an animal.

In one embodiment, a method of feeding an animal is provided. The method comprises the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain causes Enterococcus inhibition in the animal.

In another embodiment, a method of controlling a detrimental effect of Enterococcus is provided. The method comprises the steps of administering to an animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

In yet another embodiment, a method of feeding an animal is provided. The method comprises the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain prevents or reduces pododermatitis in the animal.

In still another embodiment, a method of feeding an animal is provided. The method comprises the step of administering to the animal a feed composition or drinking water comprising an effective amount of an additive comprising isolated Bacillus strains A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the EXAMPLES are applicable to any of the following embodiments of the invention described in the numbered clauses below.

1. A method of feeding an animal, the method comprising the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain causes Enterococcus inhibition in the animal.

2. The method of clause 1 wherein the animal is selected from the group consisting of a poultry species, a porcine species, a caprine species, a bovine species, an ovine species, an equine species, and a companion animal.

3. The method of clause 2 wherein the animal is a poultry species and the poultry species is a broiler chicken.

4. The method of any one of clauses 1 to 3 wherein the Enterococcus inhibition prevents Enterococcus disease in the animal.

5. The method of any one of clauses 1 to 3 wherein the Enterococcus inhibition reduces Enterococcus disease in the animal.

6. The method of any one of clauses 1 to 5 wherein the Bacillus strain produces an enzyme selected from the group consisting of an α-galactosidase, a protease, a lipase, an amylase, a xylanase, a cellulase, and combinations thereof.

7. The method of any one of clauses 1 to 6 further comprising the step of administering to the animal another bacterial strain selected from the group consisting of another Bacillus strain, a lactic acid bacterial strain, and combinations thereof.

8. The method of any one of clauses 1 to 7 wherein the strain administered is Bacillus strain A12 (NRRL No. B-67516) or a strain having all of the identifying characteristics of Bacillus strain A12 (NRRL No. B-67516).

9. The method of any one of clauses 1 to 7 wherein the strain administered is Bacillus strain 681 (NRRL No. B-67515) or a strain having all of the identifying characteristics of Bacillus strain 681 (NRRL No. B-67515).

10. The method of any one of clauses 1 to 7 wherein the strain administered is Bacillus strain 300 (NRRL No. B-50943) or a strain having all of the identifying characteristics of Bacillus strain 300 (NRRL No. B-50943).

11. The method of any one of clauses 1 to 7 wherein the strain administered is Bacillus strain 101 (NRRL No. B-67218) or a strain having all of the identifying characteristics of Bacillus strain 101 (NRRL No. B-67218).

12. The method of any one of clauses 1 to 7 wherein the strain administered is Bacillus strain 235 (NRRL No. B-67219) or a strain having all of the identifying characteristics of Bacillus strain 235 (NRRL No. B-67219).

13. The method of any one of clauses 1 to 7 wherein the strain administered is Bacillus strain 86 (NRRL No. B-50944) or a strain having all of the identifying characteristics of Bacillus strain 86 (NRRL No. B-50944).

14. The method of any one of clauses 1 to 7 wherein the strain administered is Bacillus strain 177 (NRRL No. B-67275) or a strain having all of the identifying characteristics of Bacillus strain 177 (NRRL No. B-67275).

15. The method of any one of clauses 1 to 14 wherein the Bacillus strain is administered in the feed composition at a dose of about 1.0×10³ CFU/gram of the feed composition to about 5.0×10¹² CFU/gram of the feed composition.

16. The method of any one of clauses 1 to 14 wherein the Bacillus strain is administered in the feed composition at a dose of about 1.0×10³ CFU/gram of the feed composition to about 1.0×10⁷ CFU/gram of the feed composition.

17. The method of any one of clauses 1 to 16 further comprising the step of administering an antibiotic to the animal.

18. The method of any one of clauses 1 to 17 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

19. The method of clause 18 wherein the enzyme is an NSPase or a phytase.

20. The method of any one of clauses 1 to 19 wherein the feed composition is administered daily to the animal.

21. The method of clause 1 wherein the animal is selected from the group consisting of a chicken, a pig, a horse, a pony, a cow, a turkey, a goat, a sheep, a quail, a pheasant, an ostrich, a duck, and combinations thereof.

22. The method of any one of clauses 1 to 7 or 15 to 21 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

23. A method of controlling a detrimental effect of Enterococcus, the method comprising the steps of administering to an animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

24. The method of clause 23 wherein the animal is selected from the group consisting of a poultry species, a porcine species, a caprine species, a bovine species, an ovine species, an equine species, and a companion animal.

25. The method of clause 24 wherein the animal is a poultry species and the poultry species is a broiler chicken.

26. The method of any one of clauses 23 to 25 wherein controlling the detrimental effect of Enterococcus comprises preventing Enterococcus disease in the animal.

27. The method of any one of clauses 23 to 25 wherein controlling the detrimental effect of Enterococcus comprises reducing Enterococcus disease in the animal.

28. The method of any one of clauses 23 to 27 wherein the Bacillus strain produces an enzyme selected from the group consisting of an α-galactosidase, a protease, a lipase, an amylase, a xylanase, a cellulase, and combinations thereof.

29. The method of any one of clauses 23 to 28 further comprising the step of administering to the animal another bacterial strain selected from the group consisting of another Bacillus strain, a lactic acid bacterial strain, and combinations thereof.

30. The method of any one of clauses 23 to 29 wherein the strain administered is Bacillus strain A12 (NRRL No. B-67516) or a strain having all of the identifying characteristics of Bacillus strain A12 (NRRL No. B-67516).

31. The method of any one of clauses 23 to 29 wherein the strain administered is Bacillus strain 681 (NRRL No. B-67515) or a strain having all of the identifying characteristics of Bacillus strain 681 (NRRL No. B-67515).

32. The method of any one of clauses 23 to 29 wherein the strain administered is Bacillus strain 300 (NRRL No. B-50943) or a strain having all of the identifying characteristics of Bacillus strain 300 (NRRL No. B-50943).

33. The method of any one of clauses 23 to 29 wherein the strain administered is Bacillus strain 101 (NRRL No. B-67218) or a strain having all of the identifying characteristics of Bacillus strain 101 (NRRL No. B-67218).

34. The method of any one of clauses 23 to 29 wherein the strain administered is Bacillus strain 235 (NRRL No. B-67219) or a strain having all of the identifying characteristics of Bacillus strain 235 (NRRL No. B-67219).

35. The method of any one of clauses 23 to 29 wherein the strain administered is Bacillus strain 86 (NRRL No. B-50944) or a strain having all of the identifying characteristics of Bacillus strain 86 (NRRL No. B-50944).

36. The method of any one of clauses 23 to 29 wherein the strain administered is Bacillus strain 177 (NRRL No. B-67275) or a strain having all of the identifying characteristics of Bacillus strain 177 (NRRL No. B-67275).

37. The method of any one of clauses 23 to 36 wherein the Bacillus strain is administered in the feed composition at a dose of about 1.0×10³ CFU/gram of the feed composition to about 5.0×10¹² CFU/gram of the feed composition.

38. The method of any one of clauses 23 to 36 wherein the Bacillus strain is administered in the feed composition at a dose of about 1.0×10³ CFU/gram of the feed composition to about 1.0×10⁷ CFU/gram of the feed composition.

39. The method of any one of clauses 23 to 38 further comprising the step of administering an antibiotic to the animal.

40. The method of any one of clauses 23 to 39 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

41. The method of clause 40 wherein the enzyme is an NSPase or a phytase.

42. The method of any one of clauses 23 to 41 wherein the feed composition is administered daily to the animal.

43. The method of clause 23 wherein the animal is selected from the group consisting of a chicken, a pig, a horse, a pony, a cow, a turkey, a goat, a sheep, a quail, a pheasant, an ostrich, a duck, and combinations thereof.

44. The method of any one of clauses 23 to 29 or 37 to 43 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

45. A method of feeding an animal, the method comprising the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain prevents or reduces pododermatitis in the animal.

46. The method of clause 45 wherein the animal is selected from the group consisting of a poultry species, a porcine species, a caprine species, a bovine species, an ovine species, an equine species, and a companion animal.

47. The method of clause 46 wherein the animal is a poultry species and the poultry species is a broiler chicken.

48. The method of any one of clauses 45 to 47 wherein Enterococcus disease in the animal is prevented.

49. The method of any one of clauses 45 to 47 wherein Enterococcus disease in the animal is reduced.

50. The method of any one of clauses 45 to 49 wherein the Bacillus strain produces an enzyme selected from the group consisting of an α-galactosidase, a protease, a lipase, an amylase, a xylanase, a cellulase, and combinations thereof.

51. The method of any one of clauses 45 to 50 further comprising the step of administering to the animal another bacterial strain selected from the group consisting of another Bacillus strain, a lactic acid bacterial strain, and combinations thereof.

52. The method of any one of clauses 45 to 51 wherein the strain administered is Bacillus strain A12 (NRRL No. B-67516) or a strain having all of the identifying characteristics of Bacillus strain A12 (NRRL No. B-67516).

53. The method of any one of clauses 45 to 51 wherein the strain administered is Bacillus strain 681 (NRRL No. B-67515) or a strain having all of the identifying characteristics of Bacillus strain 681 (NRRL No. B-67515).

54. The method of any one of clauses 45 to 51 wherein the strain administered is Bacillus strain 300 (NRRL No. B-50943) or a strain having all of the identifying characteristics of Bacillus strain 300 (NRRL No. B-50943).

55. The method of any one of clauses 45 to 51 wherein the strain administered is Bacillus strain 101 (NRRL No. B-67218) or a strain having all of the identifying characteristics of Bacillus strain 101 (NRRL No. B-67218).

56. The method of any one of clauses 45 to 51 wherein the strain administered is Bacillus strain 235 (NRRL No. B-67219) or a strain having all of the identifying characteristics of Bacillus strain 235 (NRRL No. B-67219).

57. The method of any one of clauses 45 to 51 wherein the strain administered is Bacillus strain 86 (NRRL No. B-50944) or a strain having all of the identifying characteristics of Bacillus strain 86 (NRRL No. B-50944).

58. The method of any one of clauses 45 to 51 wherein the strain administered is Bacillus strain 177 (NRRL No. B-67275) or a strain having all of the identifying characteristics of Bacillus strain 177 (NRRL No. B-67275).

59. The method of any one of clauses 45 to 58 wherein the Bacillus strain is administered in the feed composition at a dose of about 1.0×10³ CFU/gram of the feed composition to about 5.0×10¹² CFU/gram of the feed composition.

60. The method of any one of clauses 45 to 58 wherein the Bacillus strain is administered in the feed composition at a dose of about 1.0×10³ CFU/gram of the feed composition to about 1.0×10⁷ CFU/gram of the feed composition.

61. The method of any one of clauses 45 to 60 further comprising the step of administering an antibiotic to the animal.

62. The method of any one of clauses 45 to 61 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

63. The method of clause 62 wherein the enzyme is an NSPase or a phytase.

64. The method of any one of clauses 45 to 63 wherein the feed composition is administered daily to the animal.

65. The method of clause 45 wherein the animal is selected from the group consisting of a chicken, a pig, a horse, a pony, a cow, a turkey, a goat, a sheep, a quail, a pheasant, an ostrich, a duck, and combinations thereof.

66. The method of any one of clauses 45 to 51 or 59 to 65 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

67. A commercial package for inhibiting Enterococcus and/or preventing or reducing pododermatitis in an animal wherein the commercial package comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

68. A feed additive for an animal feed for inhibiting Enterococcus and/or preventing or reducing pododermatitis in an animal wherein the additive comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

69. An additive for the drinking water of an animal for inhibiting Enterococcus and/or preventing or reducing pododermatitis in the animal wherein the additive comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

70. An animal feed composition for inhibiting Enterococcus and/or preventing or reducing pododermatitis in an animal wherein the feed composition comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

71. The feed additive or additive for the drinking water of the animal of clause 68 or 69 in the form of a concentrate.

72. The feed additive or additive for the drinking water of the animal of clause 68 or 69 in the form of a superconcentrate.

73. The feed additive, feed composition, or additive for the drinking water of the animal of any one of clauses 68 to 72 in dry form.

74. The feed composition of clause 70 in pelleted form.

75. The commercial package of clause 67 wherein the strains are in a form selected from the group consisting of a powder, a liquid, and a pellet form.

76. The commercial package, feed additive, additive for the drinking water of the animal, or feed composition of any one of clauses 67 to 75 further comprising a carrier for the Bacillus strains.

77. The commercial package, feed additive, additive for the drinking water of the animal, or feed composition of clause 76 wherein the carrier is selected from the group consisting of a bran, rice hulls, a salt, a dextrin, and combinations thereof.

78. The commercial package, feed additive, additive for the drinking water of the animal, or feed composition of any one of clauses 67 to 77 in a bag.

79. The commercial package, feed additive, additive for the drinking water of the animal, or feed composition of clause 78 wherein the bag is a plastic bag.

80. The commercial package, feed additive, additive for the drinking water of the animal, or feed composition of any one of clauses 67 to 79 further comprising instructions for use of one or more of the Bacillus strains.

81. The commercial package, feed additive, feed composition, or additive for the drinking water of the animal of any one of clauses 67 to 80 in a 20-pound bag.

82. The commercial package, feed additive, feed composition, or additive for the drinking water of the animal of any one of clauses 67 to 80 in a 50-pound bag.

83. The feed additive or additive for the drinking water of the animal of any one of clauses 68, 69, 71 to 73, or 76 to 82 in powder form.

84. The feed additive or additive for the drinking water of the animal of any one of clauses 68, 69, 71 to 72, or 76 to 82 in liquid form.

85. The commercial package, feed additive, additive for the drinking water of the animal, or feed composition of any one of clauses 67 to 84 in a container for commercial use.

86. The commercial package, feed additive, additive for the drinking water of the animal, or feed composition of clause 85 wherein the container comprises plastic.

87. The commercial package, feed additive, additive for the drinking water of the animal, or feed composition of clause 85 wherein the container comprises paper.

88. The commercial package, feed additive, additive for the drinking water of the animal, or feed composition of any one of clauses 67 to 87 further comprising a binder.

89. The commercial package, feed additive, additive for the drinking water of the animal, or feed composition of clause 88 wherein the binder is selected from the group consisting of clay, yeast cell wall components, aluminum silicate, and glucan, or combinations thereof.

90. A method of feeding an animal, the method comprising the step of administering to the animal a feed composition or drinking water comprising an effective amount of an additive comprising isolated Bacillus strains A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

91. The method of clause 90 wherein the animal is selected from the group consisting of a poultry species, a porcine species, a caprine species, a bovine species, an ovine species, an equine species, and a companion animal.

92. The method of clause 91 wherein the animal is a poultry species and the poultry species is a broiler chicken.

93. The method of any one of clauses 90 to 92 wherein Enterococcus disease in the animal is prevented.

94. The method of any one of clauses 90 to 92 wherein Enterococcus disease in the animal is reduced.

95. The method of any one of clauses 90 to 94 wherein the Bacillus strain produces an enzyme selected from the group consisting of an α-galactosidase, a protease, a lipase, an amylase, a xylanase, a cellulase, and combinations thereof.

96. The method of any one of clauses 90 to 95 further comprising the step of administering to the animal another bacterial strain selected from the group consisting of another Bacillus strain, a lactic acid bacterial strain, and combinations thereof.

97. The method of any one of clauses 90 to 96 wherein the Bacillus strain is administered in the feed composition at a dose of about 1.0×10³ CFU/gram of the feed composition to about 5.0×10¹² CFU/gram of the feed composition.

98. The method of any one of clauses 90 to 96 wherein the Bacillus strain is administered in the feed composition at a dose of about 1.0×10³ CFU/gram of the feed composition to about 1.0×10⁷ CFU/gram of the feed composition.

99. The method of any one of clauses 90 to 98 further comprising the step of administering an antibiotic to the animal.

100. The method of any one of clauses 90 to 99 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

101. The method of clause 100 wherein the enzyme is an NSPase or a phytase.

102. The method of any one of clauses 90 to 101 wherein the feed composition is administered daily to the animal.

103. The method of clause 90 wherein the animal is selected from the group consisting of a chicken, a pig, a horse, a pony, a cow, a turkey, a goat, a sheep, a quail, a pheasant, an ostrich, a duck, and combinations thereof.

### BRIEF DESCRIPTION OF THE DRAWINGS

FIG. 1 shows broiler footpad lesion score at 42 days of age (P<0.05).

FIG. 2 shows distribution of broiler footpad lesion score at 42 days of age.

FIG. 3 shows broiler footpad lesion score at 41 days of age (P<0.05).

FIG. 4 shows distribution of broiler footpad lesion score at 41 days of age.

FIG. 5A shows the RAPD testing results for the 681 and 721 strains. FIG. 5B shows the RAPD testing results for the A12 and 54 strains. In FIG. 5A, the first lane has a molecular weight ladder and each subset of lanes represents the six different primer sets used, and within each subset, lane A represents a proprietary strain, lane B represents strain 681, and lane C represents strain 721. In FIG. 5B, each subset of lanes represents the two strains, A12 and 54, and within each subset, lanes P1-P6 represent the six different primer sets used and a molecular ladder.

FIG. 6 shows a photograph of a gel displaying RAPD PCR profiles (Primer 1 to 6) for Bacillus strains 86 and 300. Strain 86 has the top profile and strain 300 has the bottom profile. The leftmost and rightmost lanes have markers and each set of two consecutive lanes between the markers corresponds to Primers 1 to 6 going from left to right.

FIG. 7 shows a photograph of a gel displaying a RAPD PCR profile (Primer 1 to 6) for Bacillus strain 101. For Primer 1, faint bands appear at approximately 1000, 1500, and greater than 1600 base pairs. For Primer 2, an intense band appears at 650 base pairs and a faint band appears at approximately 400 base pairs. For Primer 3, a faint band appears at approximately 400 base pairs, a faint double band appears at 600 and 700 base pairs, and faint bands appear at 1000 and 1400 base pairs.

FIG. 8 shows a photograph of a gel displaying a RAPD PCR profile (Primer 2 and 3) for Bacillus strain 177. For Primer 2, intense bands appear at 600, 700, and 1000 base pairs. For Primer 3, intense bands appear at 600 base pairs, double band at 700 and 750 base pairs, double band at 900 and 1100 base pairs, and faint bands appear at 400 and greater than 1800 base pairs.

FIG. 9 shows a photograph of a gel displaying a RAPD PCR profile (Primer 1 to 6) for Bacillus strain 235. For Primer 1, faint bands appear at approximately 1000 and greater than 1600 base pairs. For Primer 2, an intense band appears at 650 base pairs and a faint band appears at approximately 400 base pairs. For Primer 3, faint bands appear at approximately 400, 600, 1000, and 1400 base pairs.

### DETAILED DESCRIPTION OF ILLUSTRATIVE EMBODIMENTS

Methods and compositions are provided for inhibiting Enterococcus in animals, such as agricultural animals. In various embodiments, the compositions for use in the methods described herein can be a commercial package, a feed additive for an animal feed composition, an additive for the drinking water of an animal, or an animal feed composition (e.g., a complete feed), each comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B-67516), a strain having all of the identifying characteristics of Bacillus strain 681 (NRRL No. B-67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, for inhibiting Enterococcus and/or preventing or reducing pododermatitis in an animal.

In one embodiment, a method of feeding an animal is provided. The method comprises the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain causes Enterococcus inhibition in the animal.

In another embodiment, a method of controlling a detrimental effect of Enterococcus is provided. The method comprises the steps of administering to an animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

In yet another embodiment, a method of feeding an animal is provided. The method comprises the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain prevents or reduces pododermatitis in the animal.

In still another embodiment, a method of feeding an animal is provided. The method comprises the step of administering to the animal a feed composition or drinking water comprising an effective amount of an additive comprising isolated Bacillus strains A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

The following clauses, and combinations thereof, provide various additional illustrative aspects of the invention described herein. The various embodiments described in this section titled “DETAILED DESCRIPTION OF ILLUSTRATIVE EMBODIMENTS” are applicable to any of the following embodiments of the invention described in the numbered clauses below.

1. A method of feeding an animal, the method comprising the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain causes Enterococcus inhibition in the animal.

2. The method of clause 1 wherein the animal is selected from the group consisting of a poultry species, a porcine species, a caprine species, a bovine species, an ovine species, an equine species, and a companion animal.

3. The method of clause 2 wherein the animal is a poultry species and the poultry species is a broiler chicken.

4. The method of any one of clauses 1 to 3 wherein the Enterococcus inhibition prevents Enterococcus disease in the animal.

5. The method of any one of clauses 1 to 3 wherein the Enterococcus inhibition reduces Enterococcus disease in the animal.

6. The method of any one of clauses 1 to 5 wherein the Bacillus strain produces an enzyme selected from the group consisting of an α-galactosidase, a protease, a lipase, an amylase, a xylanase, a cellulase, and combinations thereof.

7. The method of any one of clauses 1 to 6 further comprising the step of administering to the animal another bacterial strain selected from the group consisting of another Bacillus strain, a lactic acid bacterial strain, and combinations thereof.

8. The method of any one of clauses 1 to 7 wherein the strain administered is Bacillus strain A12 (NRRL No. B-67516) or a strain having all of the identifying characteristics of Bacillus strain A12 (NRRL No. B-67516).

9. The method of any one of clauses 1 to 7 wherein the strain administered is Bacillus strain 681 (NRRL No. B-67515) or a strain having all of the identifying characteristics of Bacillus strain 681 (NRRL No. B-67515).

10. The method of any one of clauses 1 to 7 wherein the strain administered is Bacillus strain 300 (NRRL No. B-50943) or a strain having all of the identifying characteristics of Bacillus strain 300 (NRRL No. B-50943).

11. The method of any one of clauses 1 to 7 wherein the strain administered is Bacillus strain 101 (NRRL No. B-67218) or a strain having all of the identifying characteristics of Bacillus strain 101 (NRRL No. B-67218).

12. The method of any one of clauses 1 to 7 wherein the strain administered is Bacillus strain 235 (NRRL No. B-67219) or a strain having all of the identifying characteristics of Bacillus strain 235 (NRRL No. B-67219).

13. The method of any one of clauses 1 to 7 wherein the strain administered is Bacillus strain 86 (NRRL No. B-50944) or a strain having all of the identifying characteristics of Bacillus strain 86 (NRRL No. B-50944).

14. The method of any one of clauses 1 to 7 wherein the strain administered is Bacillus strain 177 (NRRL No. B-67275) or a strain having all of the identifying characteristics of Bacillus strain 177 (NRRL No. B-67275).

15. The method of any one of clauses 1 to 14 wherein the Bacillus strain is administered in the feed composition at a dose of about 1.0×10³ CFU/gram of the feed composition to about 5.0×10¹² CFU/gram of the feed composition.

16. The method of any one of clauses 1 to 14 wherein the Bacillus strain is administered in the feed composition at a dose of about 1.0×10³ CFU/gram of the feed composition to about 1.0×10⁷ CFU/gram of the feed composition.

17. The method of any one of clauses 1 to 16 further comprising the step of administering an antibiotic to the animal.

18. The method of any one of clauses 1 to 17 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

19. The method of clause 18 wherein the enzyme is an NSPase or a phytase.

20. The method of any one of clauses 1 to 19 wherein the feed composition is administered daily to the animal.

21. The method of clause 1 wherein the animal is selected from the group consisting of a chicken, a pig, a horse, a pony, a cow, a turkey, a goat, a sheep, a quail, a pheasant, an ostrich, a duck, and combinations thereof.

22. The method of any one of clauses 1 to 7 or 15 to 21 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

23. A method of controlling a detrimental effect of Enterococcus, the method comprising the steps of administering to an animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

24. The method of clause 23 wherein the animal is selected from the group consisting of a poultry species, a porcine species, a caprine species, a bovine species, an ovine species, an equine species, and a companion animal.

25. The method of clause 24 wherein the animal is a poultry species and the poultry species is a broiler chicken.

26. The method of any one of clauses 23 to 25 wherein controlling the detrimental effect of Enterococcus comprises preventing Enterococcus disease in the animal.

27. The method of any one of clauses 23 to 25 wherein controlling the detrimental effect of Enterococcus comprises reducing Enterococcus disease in the animal.

28. The method of any one of clauses 23 to 27 wherein the Bacillus strain produces an enzyme selected from the group consisting of an α-galactosidase, a protease, a lipase, an amylase, a xylanase, a cellulase, and combinations thereof.

29. The method of any one of clauses 23 to 28 further comprising the step of administering to the animal another bacterial strain selected from the group consisting of another Bacillus strain, a lactic acid bacterial strain, and combinations thereof.

30. The method of any one of clauses 23 to 29 wherein the strain administered is Bacillus strain A12 (NRRL No. B-67516) or a strain having all of the identifying characteristics of Bacillus strain A12 (NRRL No. B-67516).

31. The method of any one of clauses 23 to 29 wherein the strain administered is Bacillus strain 681 (NRRL No. B-67515) or a strain having all of the identifying characteristics of Bacillus strain 681 (NRRL No. B-67515).

32. The method of any one of clauses 23 to 29 wherein the strain administered is Bacillus strain 300 (NRRL No. B-50943) or a strain having all of the identifying characteristics of Bacillus strain 300 (NRRL No. B-50943).

33. The method of any one of clauses 23 to 29 wherein the strain administered is Bacillus strain 101 (NRRL No. B-67218) or a strain having all of the identifying characteristics of Bacillus strain 101 (NRRL No. B-67218).

34. The method of any one of clauses 23 to 29 wherein the strain administered is Bacillus strain 235 (NRRL No. B-67219) or a strain having all of the identifying characteristics of Bacillus strain 235 (NRRL No. B-67219).

35. The method of any one of clauses 23 to 29 wherein the strain administered is Bacillus strain 86 (NRRL No. B-50944) or a strain having all of the identifying characteristics of Bacillus strain 86 (NRRL No. B-50944).

36. The method of any one of clauses 23 to 29 wherein the strain administered is Bacillus strain 177 (NRRL No. B-67275) or a strain having all of the identifying characteristics of Bacillus strain 177 (NRRL No. B-67275).

37. The method of any one of clauses 23 to 36 wherein the Bacillus strain is administered in the feed composition at a dose of about 1.0×10³ CFU/gram of the feed composition to about 5.0×10¹² CFU/gram of the feed composition.

38. The method of any one of clauses 23 to 36 wherein the Bacillus strain is administered in the feed composition at a dose of about 1.0×10³ CFU/gram of the feed composition to about 1.0×10⁷ CFU/gram of the feed composition.

39. The method of any one of clauses 23 to 38 further comprising the step of administering an antibiotic to the animal.

40. The method of any one of clauses 23 to 39 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

41. The method of clause 40 wherein the enzyme is an NSPase or a phytase.

42. The method of any one of clauses 23 to 41 wherein the feed composition is administered daily to the animal.

43. The method of clause 23 wherein the animal is selected from the group consisting of a chicken, a pig, a horse, a pony, a cow, a turkey, a goat, a sheep, a quail, a pheasant, an ostrich, a duck, and combinations thereof.

44. The method of any one of clauses 23 to 29 or 37 to 43 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

45. A method of feeding an animal, the method comprising the step of administering to the animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, wherein the Bacillus strain prevents or reduces pododermatitis in the animal.

46. The method of clause 45 wherein the animal is selected from the group consisting of a poultry species, a porcine species, a caprine species, a bovine species, an ovine species, an equine species, and a companion animal.

47. The method of clause 46 wherein the animal is a poultry species and the poultry species is a broiler chicken.

48. The method of any one of clauses 45 to 47 wherein Enterococcus disease in the animal is prevented.

49. The method of any one of clauses 45 to 47 wherein Enterococcus disease in the animal is reduced.

50. The method of any one of clauses 45 to 49 wherein the Bacillus strain produces an enzyme selected from the group consisting of an α-galactosidase, a protease, a lipase, an amylase, a xylanase, a cellulase, and combinations thereof.

51. The method of any one of clauses 45 to 50 further comprising the step of administering to the animal another bacterial strain selected from the group consisting of another Bacillus strain, a lactic acid bacterial strain, and combinations thereof.

52. The method of any one of clauses 45 to 51 wherein the strain administered is Bacillus strain A12 (NRRL No. B-67516) or a strain having all of the identifying characteristics of Bacillus strain A12 (NRRL No. B-67516).

53. The method of any one of clauses 45 to 51 wherein the strain administered is Bacillus strain 681 (NRRL No. B-67515) or a strain having all of the identifying characteristics of Bacillus strain 681 (NRRL No. B-67515).

54. The method of any one of clauses 45 to 51 wherein the strain administered is Bacillus strain 300 (NRRL No. B-50943) or a strain having all of the identifying characteristics of Bacillus strain 300 (NRRL No. B-50943).

55. The method of any one of clauses 45 to 51 wherein the strain administered is Bacillus strain 101 (NRRL No. B-67218) or a strain having all of the identifying characteristics of Bacillus strain 101 (NRRL No. B-67218).

56. The method of any one of clauses 45 to 51 wherein the strain administered is Bacillus strain 235 (NRRL No. B-67219) or a strain having all of the identifying characteristics of Bacillus strain 235 (NRRL No. B-67219).

57. The method of any one of clauses 45 to 51 wherein the strain administered is Bacillus strain 86 (NRRL No. B-50944) or a strain having all of the identifying characteristics of Bacillus strain 86 (NRRL No. B-50944).

58. The method of any one of clauses 45 to 51 wherein the strain administered is Bacillus strain 177 (NRRL No. B-67275) or a strain having all of the identifying characteristics of Bacillus strain 177 (NRRL No. B-67275).

59. The method of any one of clauses 45 to 58 wherein the Bacillus strain is administered in the feed composition at a dose of about 1.0×10³ CFU/gram of the feed composition to about 5.0×10¹² CFU/gram of the feed composition.

60. The method of any one of clauses 45 to 58 wherein the Bacillus strain is administered in the feed composition at a dose of about 1.0×10³ CFU/gram of the feed composition to about 1.0×10⁷ CFU/gram of the feed composition.

61. The method of any one of clauses 45 to 60 further comprising the step of administering an antibiotic to the animal.

62. The method of any one of clauses 45 to 61 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

63. The method of clause 62 wherein the enzyme is an NSPase or a phytase.

64. The method of any one of clauses 45 to 63 wherein the feed composition is administered daily to the animal.

65. The method of clause 45 wherein the animal is selected from the group consisting of a chicken, a pig, a horse, a pony, a cow, a turkey, a goat, a sheep, a quail, a pheasant, an ostrich, a duck, and combinations thereof.

66. The method of any one of clauses 45 to 51 or 59 to 65 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

67. A commercial package for inhibiting Enterococcus and/or preventing or reducing pododermatitis in an animal wherein the commercial package comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

68. A feed additive for an animal feed for inhibiting Enterococcus and/or preventing or reducing pododermatitis in an animal wherein the additive comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

69. An additive for the drinking water of an animal for inhibiting Enterococcus and/or preventing or reducing pododermatitis in the animal wherein the additive comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

70. An animal feed composition for inhibiting Enterococcus and/or preventing or reducing pododermatitis in an animal wherein the feed composition comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

71. The feed additive or additive for the drinking water of the animal of clause 68 or 69 in the form of a concentrate.

72. The feed additive or additive for the drinking water of the animal of clause 68 or 69 in the form of a superconcentrate.

73. The feed additive, feed composition, or additive for the drinking water of the animal of any one of clauses 68 to 72 in dry form.

74. The feed composition of clause 70 in pelleted form.

75. The commercial package of clause 67 wherein the strains are in a form selected from the group consisting of a powder, a liquid, and a pellet form.

76. The commercial package, feed additive, additive for the drinking water of the animal, or feed composition of any one of clauses 67 to 75 further comprising a carrier for the Bacillus strains.

77. The commercial package, feed additive, additive for the drinking water of the animal, or feed composition of clause 76 wherein the carrier is selected from the group consisting of a bran, rice hulls, a salt, a dextrin, and combinations thereof.

78. The commercial package, feed additive, additive for the drinking water of the animal, or feed composition of any one of clauses 67 to 77 in a bag.

79. The commercial package, feed additive, additive for the drinking water of the animal, or feed composition of clause 78 wherein the bag is a plastic bag.

80. The commercial package, feed additive, additive for the drinking water of the animal, or feed composition of any one of clauses 67 to 79 further comprising instructions for use of one or more of the Bacillus strains.

81. The commercial package, feed additive, feed composition, or additive for the drinking water of the animal of any one of clauses 67 to 80 in a 20-pound bag.

82. The commercial package, feed additive, feed composition, or additive for the drinking water of the animal of any one of clauses 67 to 80 in a 50-pound bag.

83. The feed additive or additive for the drinking water of the animal of any one of clauses 68, 69, 71 to 73, or 76 to 82 in powder form.

84. The feed additive or additive for the drinking water of the animal of any one of clauses 68, 69, 71 to 72, or 76 to 82 in liquid form.

85. The commercial package, feed additive, additive for the drinking water of the animal, or feed composition of any one of clauses 67 to 84 in a container for commercial use.

86. The commercial package, feed additive, additive for the drinking water of the animal, or feed composition of clause 85 wherein the container comprises plastic.

87. The commercial package, feed additive, additive for the drinking water of the animal, or feed composition of clause 85 wherein the container comprises paper.

88. The commercial package, feed additive, additive for the drinking water of the animal, or feed composition of any one of clauses 67 to 87 further comprising a binder.

89. The commercial package, feed additive, additive for the drinking water of the animal, or feed composition of clause 88 wherein the binder is selected from the group consisting of clay, yeast cell wall components, aluminum silicate, and glucan, or combinations thereof.

90. A method of feeding an animal, the method comprising the step of administering to the animal a feed composition or drinking water comprising an effective amount of an additive comprising isolated Bacillus strains A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

91. The method of clause 90 wherein the animal is selected from the group consisting of a poultry species, a porcine species, a caprine species, a bovine species, an ovine species, an equine species, and a companion animal.

92. The method of clause 91 wherein the animal is a poultry species and the poultry species is a broiler chicken.

93. The method of any one of clauses 90 to 92 wherein Enterococcus disease in the animal is prevented.

94. The method of any one of clauses 90 to 92 wherein Enterococcus disease in the animal is reduced.

95. The method of any one of clauses 90 to 94 wherein the Bacillus strain produces an enzyme selected from the group consisting of an α-galactosidase, a protease, a lipase, an amylase, a xylanase, a cellulase, and combinations thereof.

96. The method of any one of clauses 90 to 95 further comprising the step of administering to the animal another bacterial strain selected from the group consisting of another Bacillus strain, a lactic acid bacterial strain, and combinations thereof.

97. The method of any one of clauses 90 to 96 wherein the Bacillus strain is administered in the feed composition at a dose of about 1.0×10³ CFU/gram of the feed composition to about 5.0×10¹² CFU/gram of the feed composition.

98. The method of any one of clauses 90 to 96 wherein the Bacillus strain is administered in the feed composition at a dose of about 1.0×10³ CFU/gram of the feed composition to about 1.0×10⁷ CFU/gram of the feed composition.

99. The method of any one of clauses 90 to 98 further comprising the step of administering an antibiotic to the animal.

100. The method of any one of clauses 90 to 99 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.

101. The method of clause 100 wherein the enzyme is an NSPase or a phytase.

102. The method of any one of clauses 90 to 101 wherein the feed composition is administered daily to the animal.

103. The method of clause 90 wherein the animal is selected from the group consisting of a chicken, a pig, a horse, a pony, a cow, a turkey, a goat, a sheep, a quail, a pheasant, an ostrich, a duck, and combinations thereof.

In various embodiments, the animal to which a feed additive, a feed composition, or drinking water as described herein is administered can be selected from the group consisting of a poultry species, a porcine species, a caprine species, a bovine species, an ovine species, an equine species, and a companion animal. In the embodiment where the animal is a companion animal, the companion animal can be, for example, a canine species or a feline species. In the embodiment where the animal is a porcine species, the porcine species can be selected from the group consisting of a grow finish pig, a nursery pig, a sow, and a breeding stock pig. In various exemplary embodiments, the animal can be selected from the group consisting of a chicken (e.g., a broiler or a layer), a pig, a horse, a pony, a cow, a turkey, a goat, a sheep, a quail, a pheasant, an ostrich, a duck, and combinations thereof. In another embodiment, the feed additive, feed composition, or drinking water as described herein could be administered to a human.

In another embodiment, Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, can reduce contamination by detrimental bacteria of meat products, including animal feet, produced from the animal.

In various embodiments of the methods, commercial package, feed additive, feed composition, or additive for drinking water described herein, the Bacillus strain can have an effect selected from the group consisting of maintaining microbial balance in the gut of the animal, preventing or reducing Enterococcus disease in the animal (e.g., pododermatitis, endocarditis, arthritis, osteomyelitis, spondylitis, and femoral head necrosis, for example, in broilers or other agricultural or companion animals), improving animal performance or health, maintaining gut health in the animal, reducing detrimental pathogens in the gut of the animal, odor reduction, reducing detrimental pathogens in the urine or feces of the animal, and preserving the growth of beneficial bacteria in the gut of the animal.

In any of the embodiments, described herein the Bacillus strain can be a Bacillus subtilis strain (e.g., strains 86, 300, 101, 235, A12, and 681), or a Bacillus pumilus strain (e.g., strain 177). In one embodiment, the Bacillus strain produces an enzyme selected from the group consisting of an α-galactosidase, a protease, a lipase, an amylase, a xylanase, a cellulase, and combinations thereof.

In any embodiments described herein, the Bacillus strains can be administered alone or in any combination, or can be in the form of any composition described herein so that the strains are alone or in any combination in the compositions described herein. The Bacillus strains described herein can also be used in combination with other microbial strains, including other Bacillus strains or Lactobacillus strains.

In one embodiment of the invention, an effective amount of the Bacillus strain can be administered to inhibit Enterococcus disease in the animal. As used herein, “inhibit Enterococcus” disease can mean reducing Enterococcus disease (e.g., pododermatitis, endocarditis, arthritis, osteomyelitis, spondylitis, and femoral head necrosis, for example, in broilers or other agricultural or companion animals), preventing Enterococcus disease, maintaining the normal microbial balance in the animal, reducing the number of detrimental Enterococci in the animal, reducing the activity of Enterococci in the animal, or reducing the symptoms of Enterococcus disease in the animal, or combinations thereof. By “effective amount” is meant an amount of the Bacillus strain (e.g., strain A12, 681, 86, 300, 101, 177, or 235) capable of Enterococcus inhibition or capable of controlling a detrimental effect of Enterococcus, as described above, by any mechanism.

For methods involving reducing or preventing pododermatitis in the animal, “reducing,” “reduces,” “reduce”, “preventing,” “prevents,” or “prevent” can mean reducing pododermatitis, preventing pododermatitis, or reducing the symptoms of pododermatitis in the animal, or combinations thereof. For methods involving reducing or preventing pododermatitis in the animal an “effective amount” of the Bacillus strain means an amount capable of reducing or preventing pododermatitis, by any mechanism. In other embodiments, an “effective amount” of the Bacillus strain means an amount capable of controlling a detrimental effect of Enterococcus, by any mechanism, wherein the detrimental effect can be pododermatitis, for example.

In the embodiments, involving “controlling a detrimental effect of Enterococcus,” “controlling a detrimental effect of Enterococcus” can mean reducing Enterococcus disease (e.g., pododermatitis, endocarditis, arthritis, osteomyelitis, spondylitis, and femoral head necrosis, for example, in broilers or other agricultural or companion animals), preventing Enterococcus disease, maintaining the normal microbial balance in the animal, reducing the number of detrimental Enterococci in the animal, reducing the activity of Enterococci in the animal, or reducing the symptoms of Enterococcus disease in the animal, or combinations thereof. By “effective amount” is meant an amount of the Bacillus strain (e.g., Bacillus strain A12, and/or Bacillus strain 681, and/or Bacillus strain 300, and/or Bacillus strain 101, and/or Bacillus strain 235, and/or Bacillus strain 86, and/or Bacillus strain 177) capable of controlling a detrimental effect of Enterococcus, as described above, by any mechanism.

In embodiments described herein wherein the compositions of the present invention comprising Bacillus strains A12, and/or 681, and/or 300, and/or 101, and/or 235, and/or 86, and/or 177, or strains having their identifying characteristics, are administered to an animal, the compositions are preferably administered to animals orally in a feed composition or in drinking water, but any effective method of administration known to those skilled in the art may be utilized such as in a paste, a liquid drench, a top dress, or a capsule. In one illustrative embodiment, the Bacillus strains A12, and/or 681, and/or 300, and/or 101, and/or 235, and/or 86, and/or 177, or strains having their identifying characteristics, are provided in the form of an additive for addition to the drinking water of an animal. In another embodiment, the strains may be used as a litter treatment.

In another illustrative embodiment, the Bacillus strains A12, and/or 681, and/or 300, and/or 101, and/or 235, and/or 86, and/or 177, or strains having their identifying characteristics, are provided in the form of a feed additive for addition to a feed composition. The feed composition may contain Bacillus strain A12, and/or 681, and/or 300, and/or 101, and/or 235, and/or 86, and/or 177, or strains having their identifying characteristics, in a mixture with an animal feed blend, including any art-recognized animal feed blend or any animal feed blend described herein. As used herein, “feed composition” or “animal feed composition” means a feed composition comprising Bacillus strain A12, and/or 681, and/or 300, and/or 101, and/or 235, and/or 86, and/or 177, or strains having their identifying characteristics, in a mixture with an animal feed blend, and, optionally any other components that could be used in a feed composition, including other bacterial strains, such as other Bacillus strains or Lactobacillus strains. In one embodiment, the feed composition may be in the form of a ground meal.

Any animal feed blend, including those known in the art and those described herein, may be used in accordance with the methods and compositions described in this patent application, such as rapeseed meal, cottonseed meal, soybean meal, cornmeal, barley, wheat, silage, and haylage. In various embodiments, the animal feed blend can be supplemented with Bacillus strain A12, and/or 681, and/or 300, and/or 101, and/or 235, and/or 86, and/or 177, or strains having their identifying characteristics, but other ingredients may optionally be added to the animal feed blend, including other bacterial strains, such as other Bacillus strains or Lactobacillus strains.

In various illustrative embodiments, optional ingredients of the animal feed blend include sugars and complex carbohydrates such as both water-soluble and water-insoluble monosaccharides, disaccharides, and polysaccharides. Other optional ingredients include dried distillers grain solubles, fat (e.g., crude fat), phosphorous, sodium bicarbonate, limestone, salt, phytate, calcium, sodium, sulfur, magnesium, potassium, copper, iron, manganese, zinc, ash, fish oil, an oil derived from fish meal, raw seed (e.g., flaxseed), an antioxidant, and starch. In another embodiment, minerals may be added in the form of a mineral premix.

Optional amino acid ingredients that may be added to the animal feed blend are arginine, histidine, isoleucine, leucine, lysine, cysteine, methionine, phenylalanine, threonine, tryptophan, valine, tyrosine ethyl HCl, alanine, aspartic acid, sodium glutamate, glycine, proline, serine, cysteine ethyl HCl, and analogs, and salts thereof. Vitamins that may be optionally added are thiamine HCl, riboflavin, pyridoxine HCl, niacin, niacinamide, inositol, choline chloride, calcium pantothenate, biotin, folic acid, ascorbic acid, and vitamins A, B, K, D, E, and the like. In another embodiment, vitamins may be added in the form of a vitamin premix. In yet another embodiment, protein ingredients may be added to the animal feed blend and include protein obtained from meat meal, bone meal, or fish meal, liquid or powdered egg, fish solubles, crude protein, and the like.

In another illustrative aspect, any medicament ingredients known in the art may be added to the animal feed blend or to an additive for the drinking water of the animal, such as antibiotics. In various embodiments, the antibiotic is selected from the group consisting of ampicillin, chloramphenicol, ciprofloxacin, clindamycin, tetracycline, chlortetracycline, Denagard™ (i.e., tiamulin), BMD™ (i.e., bacitracin methylene disalicylate), Carbadox™ (i.e., carbadox), Stafac™ (i.e., virginiamycin), erythromycin, levofloxacin, trimethoprim/sulfamethoxazole, trimethoprim, daptomycin, rifampicin, Tylan™ (i.e., tylosin), Pulmotil™ (i.e., tilmicosin), vancomycin, and combinations thereof. In another embodiment, the animal feed blend, the feed composition, the feed additive, or the additive for the drinking water of the animal may contain no antibiotics.

In another illustrative embodiment, one or more enzymes may be added to the animal feed blend. In various embodiments, the enzymes that may be added include a galactosidase, a phytase, a protease, a lipase, an amylase, a hemicellulase, an arabinoxylanase, a xylanase, a cellulase, an NSPase, combinations thereof, and any other enzyme that improves the effectiveness of the feed composition for Enterococcus inhibition or controlling a detrimental effect of Enterococcus, or preventing or reducing pododermatitis in the animal, or controlling detrimental effects of pododermatitis in the animal. In yet another embodiment, yeast, fungi (e.g., Aspergillus or Trichoderma), or micronutrients may be added to the animal feed. Any of the ingredients described above that are suitable for addition to an additive for the drinking water of the animal may be added as a component of the additive for the drinking water of the animal as described herein.

In various illustrative embodiments, the Bacillus strain (e.g., Bacillus strain A12, and/or 681, and/or 300, and/or 101, and/or 235, and/or 86, and/or 177, or strains having their identifying characteristics), or any other bacterial strains added in addition to Bacillus strain A12, and/or 681, and/or 300, and/or 101, and/or 235, and/or 86, and/or 177, or strains having their identifying characteristics, can be administered in the feed composition at a dose of about 1.0×10³ CFU/gram of the feed composition to about 5.0×10¹² CFU/gram of the feed composition or at a dose of about 1.0×10³ CFU/gram of the feed composition to about 1.0×10⁷ CFU/gram of the feed composition. In other embodiments, the Bacillus strain (e.g., Bacillus strain A12, and/or 681, and/or 300, and/or 101, and/or 235, and/or 86, and/or 177, or strains having their identifying characteristics) is administered in the feed composition at a dose greater than about 1.0×10³ CFU/gram of the feed composition, at a dose greater than about 1.1×10³ CFU/gram of the feed composition, at a dose greater than about 1.25×10³ CFU/gram of the feed composition, at a dose greater than about 1.5×10³ CFU/gram of the feed composition, at a dose greater than about 1.75×10³ CFU/gram of the feed composition, at a dose greater than about 1.0×10⁴ CFU/gram of the feed composition, at a dose greater than about 2.0×10⁴ CFU/gram of the feed composition, at a dose greater than about 3.0×10⁴ CFU/gram of the feed composition, at a dose greater than about 4.0×10⁴ CFU/gram of the feed composition, at a dose greater than about 5.0×10⁴ CFU/gram of the feed composition, at a dose greater than about 6.0×10⁴ CFU/gram of the feed composition, at a dose greater than about 7.0×10⁴ CFU/gram of the feed composition, at a dose greater than about 8.0×10⁴ CFU/gram of the feed composition, at a dose greater than about 1.0×10⁵ CFU/gram of the feed composition, at a dose greater than about 1.0×10⁶ CFU/gram of the feed composition, at a dose greater than about 1.0×10⁷ CFU/gram of the feed composition, at a dose greater than about 1.0×10⁸ CFU/gram of the feed composition, at a dose greater than about 1.0×10⁹ CFU/gram of the feed composition, at a dose greater than about 1.0×10¹⁰ CFU/gram of the feed composition, at a dose greater than about 1.0×10¹¹ CFU/gram of the feed composition, or at a dose greater than about 1.0×10¹² CFU/gram of the feed composition. In yet another embodiment, the Bacillus strain (e.g., Bacillus strain A12, and/or 681, and/or 300, and/or 101, and/or 235, and/or 86, and/or 177, or strains having their identifying characteristics) is administered in the feed composition at a dose of about 7.3×10⁴ CFU/gram of the feed composition. In another embodiment, the Bacillus strain (e.g., Bacillus strain A12, and/or 681, and/or 300, and/or 101, and/or 235, and/or 86, and/or 177, or strains having their identifying characteristics), or any other bacterial strains added in addition to Bacillus strain A12, and/or 681, and/or 300, and/or 101, and/or 235, and/or 86, and/or 177, or strains having their identifying characteristics, can be administered in the feed composition at a dose of about 1.0×10² CFU/gram of the feed composition to about 5.0×10¹² CFU/gram of the feed composition or at a dose of about 1.0×10² CFU/gram of the feed composition to about 1.0×10⁷ CFU/gram of the feed composition, or at a dose greater than about 1.0×10² CFU/gram of the feed composition. In another embodiment, any of the dosages described herein can be in CFU/ml of drinking water in embodiments where the strains are administered in the drinking water of the animal.

In various embodiments, the Bacillus strain (e.g., Bacillus strain A12, and/or 681, and/or 300, and/or 101, and/or 235, and/or 86, and/or 177, or strains having their identifying characteristics) for use in accordance with the methods and compositions described herein can be selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275), a strain having all of the identifying characteristics of Bacillus strain A12 (NRRL No. B-67516), a strain having all of the identifying characteristics of Bacillus strain 681 (NRRL No. B-67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof. Bacillus strains A12 and 681 were deposited on Sep. 14, 2017 at the Agricultural Research Service Culture Collection (NRRL), National Center for Agricultural Utilization Research, Agricultural Research Service, USDA, 1815 North University Street, Peoria, Illinois 61604-3999, and were given accession numbers B-67516 and B-67515, respectively. The deposits were made under the provisions of the Budapest Treaty on the International Recognition of the Deposit of Microorganisms for the Purposes of Patent Procedure. The NRRL strain designations are B-67516 and B-67515 which are equivalent to strain or Bacillus strain A12 and 681, respectively, as referred to in the application. Bacillus strain MDG86 and Bacillus strain MDG300 were deposited on Mar. 14, 2014 at the Agricultural Research Service Culture Collection (NRRL), National Center for Agricultural Utilization Research, Agricultural Research Service, USDA, 1815 North University Street, Peoria, Illinois 61604-3999, and were given accession numbers B-50944 and B-50943, respectively. The deposits were made under the provisions of the Budapest Treaty on the International Recognition of the Deposit of Microorganisms for the Purposes of Patent Procedure. The NRRL strain designations are MDG86 and MDG300, which are equivalent to strain or Bacillus strain 86 and 300, respectively, as referred to in the application. Bacillus strain MDG 101 and Bacillus strain MDG 235 were deposited on Jan. 4, 2016 at the Agricultural Research Service Culture Collection (NRRL), National Center for Agricultural Utilization Research, Agricultural Research Service, USDA, 1815 North University Street, Peoria, Illinois 61604-3999, and were given accession numbers B-67218 and B-67219, respectively. The deposit was made under the provisions of the Budapest Treaty on the International Recognition of the Deposit of Microorganisms for the Purposes of Patent Procedure. Bacillus strain MGL177 was deposited on Jun. 7, 2016 at the Agricultural Research Service Culture Collection (NRRL), National Center for Agricultural Utilization Research, Agricultural Research Service, USDA, 1815 North University Street, Peoria, Illinois 61604-3999, and was given accession number B-67275. The deposit was made under the provisions of the Budapest Treaty on the International Recognition of the Deposit of Microorganisms for the Purposes of Patent Procedure. The NRRL strain designations are MDG 101, MDG 235, and MGL177 which are equivalent to strain or Bacillus strain 101, 235, and 177, respectively, as referred to in the application.

Any of these strains can be administered alone or in combination in the form of a feed composition (e.g., a complete feed comprising an animal feed blend) or drinking water for an animal. In one embodiment, multiple strains are administered in combination in a single composition. In another embodiment, multiple strains are administered in combination in separate compositions.

In another embodiment, one or more of the Bacillus strains described in the preceding paragraphs (e.g., Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains having their identifying characteristics) can be administered to the animal along with another bacterial strain selected from the group consisting of another Bacillus strain, a lactic acid bacterial strain, and combinations thereof. In yet another embodiment, one or more of the Bacillus strains described in the preceding paragraphs (e.g., Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains having their identifying characteristics) can be administered to the animal along with any other bacterial strain effective to inhibit or control detrimental effects of Enterococcus in the animal, or prevent or reduce pododermatitis or control detrimental effects of Enterococcus to control pododermatitis in the animal.

As used herein “a strain having all of the identifying characteristics of” Bacillus strain A12, and/or Bacillus strain 681, and/or Bacillus strain 300, and/or Bacillus strain 101, and/or Bacillus strain 235, and/or Bacillus strain 86, and/or Bacillus strain 177 can be a mutant strain having all of the identifying characteristics of Bacillus strain A12, and/or Bacillus strain 681, and/or Bacillus strain 300, and/or Bacillus strain 101, and/or Bacillus strain 235, and/or Bacillus strain 86, and/or Bacillus strain 177 (e.g., a DNA fingerprint based on DNA analysis that corresponds to the DNA fingerprint of Bacillus strain A12, and/or Bacillus strain 681, and/or Bacillus strain 300, and/or Bacillus strain 101, and/or Bacillus strain 235, and/or Bacillus strain 86, and/or Bacillus strain 177, enzyme activities that correspond to Bacillus strain A12, and/or Bacillus strain 681, and/or Bacillus strain 300, and/or Bacillus strain 101, and/or Bacillus strain 235, and/or Bacillus strain 86, and/or Bacillus strain 177, antimicrobial activity that corresponds to Bacillus strain A12, and/or Bacillus strain 681, and/or Bacillus strain 300, and/or Bacillus strain 101, and/or Bacillus strain 235, and/or Bacillus strain 86, and/or Bacillus strain 177, antibiotic sensitivity and tolerance profiles that correspond to Bacillus strain A12, and/or Bacillus strain 681, and/or Bacillus strain 300, and/or Bacillus strain 101, and/or Bacillus strain 235, and/or Bacillus strain 86, and/or Bacillus strain 177, or combinations of these identifying characteristics). In alternate embodiments, the mutation can be a natural mutation, or a genetically engineered mutation. In another embodiment, “a strain having all of the identifying characteristics of” Bacillus strain A12, and/or Bacillus strain 681, and/or Bacillus strain 300, and/or Bacillus strain 101, and/or Bacillus strain 235, and/or Bacillus strain 86, and/or Bacillus strain 177, can be a strain, for example, produced by isolating one or more plasmids from Bacillus strain A12, and/or Bacillus strain 681, and/or Bacillus strain 300, and/or Bacillus strain 101, and/or Bacillus strain 235, and/or Bacillus strain 86, and/or Bacillus strain 177, and introducing the one or more plasmids into another bacterium, such as another Bacillus strain, as long as the one or more plasmids contain DNA that provides the identifying characteristics of Bacillus strain A12, and/or Bacillus strain 681, and/or Bacillus strain 300, and/or Bacillus strain 101, and/or Bacillus strain 235, and/or Bacillus strain 86, and/or Bacillus strain 177 (e.g., a DNA fingerprint based on DNA analysis that corresponds to the DNA fingerprint of Bacillus strain A12, and/or Bacillus strain 681, and/or Bacillus strain 300, and/or Bacillus strain 101, and/or Bacillus strain 235, and/or Bacillus strain 86, and/or Bacillus strain 177).

The feed composition or drinking water comprising Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, may be administered to the animal for any time period that is effective to inhibit Enterococcus or control a detrimental effect of Enterococcus, or reduce or prevent pododermatitis or control a detrimental effect of Enterococcus to control pododermatitis in the animal, or combinations thereof. For example, in one embodiment the feed composition or drinking water may be provided to the animal daily. The time periods for administration of the feed composition or drinking water are non-limiting and it should be appreciated that any time period or administration schedule determined to be effective to inhibit Enterococcus or control a detrimental effect of Enterococcus, or reduce or prevent pododermatitis or control a detrimental effect of Enterococcus to control pododermatitis in the animal, or combinations thereof, may be used.

In another embodiment an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, can be used to reduce detrimental bacterial contaminants in meat produced from the animal, including the paws of the animal.

In one embodiment, the feed additive for addition to an animal feed blend to produce a complete feed composition can be mixed with the animal feed blend, for example, with an automated micro-nutrient delivery system, or, for example, by hand-weighing and addition to achieve any of the doses of Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, described herein, for administration to the animal in the form of a complete feed composition. The mixing can also be done by any other suitable method known in the art for combining direct-fed microbials with an animal feed blend to obtain a uniform mixture. In various embodiments, the mixing can be done for any suitable time period (e.g., about 1 to about 4 minutes). In the embodiment where the Bacillus strain A12, and/or Bacillus strain 681, and/or Bacillus strain 300, and/or Bacillus strain 101, and/or Bacillus strain 235, and/or Bacillus strain 86, and/or Bacillus strain 177, or a strain with their identifying characteristics, is in the form of an additive for the drinking water of the animal, the Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, can be in the form of, for example, a powder, a liquid, or pellets, and can be mixed with the drinking water using any suitable method known in the art to achieve any of the doses of Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, described herein, for administration to the animal in the drinking water of the animal. Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, can also be fed directly to the animal orally (i.e., by oral insertion) in the form of a powder, a liquid, or a pellet.

In any of the composition embodiments described herein, Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, can be added to the compositions to reduce or prevent pododermatitis in the animal, or to control a detrimental effect of or to inhibit Enterococcus. In one embodiment, the control or inhibition of Enterococcus is to control pododermatitis in the animal.

In any of the composition embodiments described herein, Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof, can cause an effect selected from the group consisting of inhibiting Enterococcus or controlling a detrimental effect of Enterococcus in the animal. These effects are non-limiting examples of the types of effects these Bacillus strains can cause. For example, in various composition embodiments of the methods, commercial package, feed additive, feed composition, or additive for drinking water described herein, the Bacillus strain can have an effect selected from the group consisting of maintaining microbial balance in the gut of the animal, preventing or reducing Enterococcus disease in the animal (e.g., pododermatitis, endocarditis, arthritis, osteomyelitis, spondylitis, and femoral head necrosis, for example, in broilers or other agricultural or companion animals), improving animal performance or health, maintaining gut health in the animal, reducing detrimental pathogens in the gut of the animal, odor reduction, reducing detrimental pathogens in the urine or feces of the animal, reducing contamination by detrimental bacteria of meat products produced from the animal, and preserving the growth of beneficial bacteria in the gut of the animal.

In one illustrative aspect, the feed additive, additive for the drinking water of the animal, or the feed composition can be in the form of a commercial package. In another illustrative embodiment, the feed additive or additive for the drinking water of the animal, or the Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, in the commercial package can be in the form of a concentrate (e.g., about 1×10⁸ to about 5×10⁹ CFU/g) or a superconcentrate (e.g., about 1×10¹⁰ to about 5×10¹² CFU/g). In another embodiment, the feed additive, feed composition, or additive for the drinking water of the animal, or the Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, in a composition in a commercial package, can be in a dry form (e.g., a powder), a pelleted form, a liquid form, in the form of a top-dressing, or in the form of a gel, or any other suitable form.

In yet another embodiment, the strains in the form of a commercial package can be, for example, in a dry form (e.g., a powder or freeze-dried form), in a pelleted form, or in a liquid form.

In another illustrative embodiment, the commercial package, feed additive, additive for the drinking water of the animal, or feed composition can further comprise a carrier for the Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics. The carrier can be selected from the group consisting of a bran, rice hulls, a salt, mineral oil, a dextrin (e.g., maltodextrin), whey, sugar, sucrose, limestone, yeast culture, dried starch, sodium silico aluminate, silicon dioxide, polypropylene glycol, polysorbate 80, vegetable oil, and combinations thereof. In another embodiment, the carrier can be any suitable carrier known in the art for a direct-fed microbial. In another embodiment, the commercial package, feed additive, additive for the drinking water of the animal, or feed composition can further comprise a binder such as clay, yeast cell wall components, aluminum silicate, glucan, or other known binders. In another embodiment, the commercial package, feed additive, additive for the drinking water of the animal, or feed composition can further comprise inorganic/organic binders, essential oils, and/or organic acids.

In yet other embodiments, the commercial package, feed additive, additive for the drinking water of the animal, or feed composition comprising Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics is in a container for commercial use. In various embodiments the container can be, for example, a bag (e.g., a 20-pound bag, a 50-pound bag, a 2-ounce bag, a 1-pound bag, or a 1-kilogram bag), a pouch, a drum, a bottle, or a box. In illustrative aspects, the container for the commercial package, feed additive, additive for the drinking water of the animal, or feed composition comprising Bacillus strain A12, and/or Bacillus strain 681, and/or Bacillus strain 300, and/or Bacillus strain 101, and/or Bacillus strain 235, and/or Bacillus strain 86, and/or Bacillus strain 177, or strains with their identifying characteristics, can comprise plastic, metal, foil, paper, fiber, or cardboard (e.g., a plastic pail, a paper bag, a foil bag, a fiber drum, etc.). The commercial package, feed additive, additive for the drinking water of the animal, or feed composition can further comprise instructions for use of one or more of the Bacillus strains.

The following examples are for illustrative purposes only. The examples are non-limiting, and are not intended to limit the invention in any way.

### Example 1

### In Vivo Studies of Efficacy of Bacillus Strains 681, A12, 101, 235, 177, 86, and 300 Against Footpad Lesions in Broiler Chickens

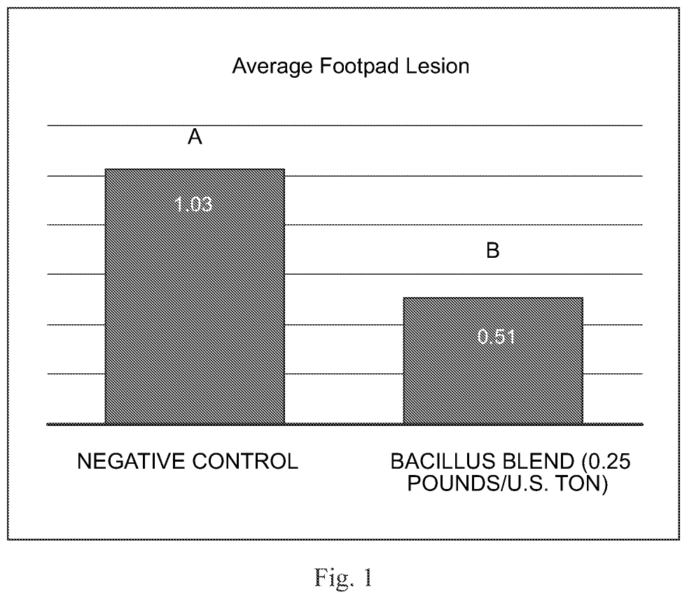

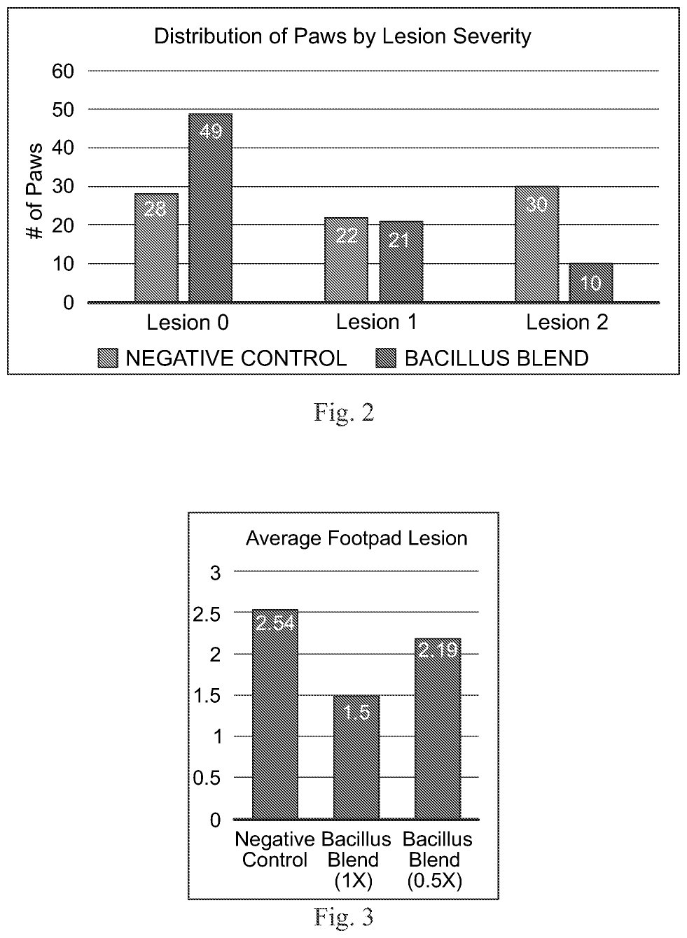

In trial 1, the efficacy of a Bacillus strain combination in reducing the severity of footpad lesions was determined in a floor pen study using broiler chickens exposed to new litter. This trial used 400 Ross×Ross birds per treatment (8 reps of 50 birds per pen). Control birds without supplementation were compared to birds supplemented with a combination of Bacillus strains A12 (NRRL No. B-67516), 681 (NRRL No. B-67515), 101 (NRRL No. B-67218), 235 (NRRL No. B-67219), 177 (NRRL No. B-67275), 86 (NRRL No. B-50944), and 300 (NRRL No. B-50943) in the feed at 0.25 pounds/U.S ton from starter to finisher stage (day 0 to 42). At day 42, 5 birds were randomly selected from each pen and evaluated for footpad lesion severity using a scale of 0-2. Bacillus blend supplementation resulted in decreased lesion severity (0.51 compared to 1.03, P<0.05; FIG. 1 and Table 1) and resulted in more birds with lesion score 0 category (49 compared to 28; FIG. 2), providing evidence for this Bacillus strain blend's effect on broiler health and welfare and its utility for operations struggling with prevention and treatment of pododermatitis.

TABLE 1
Average Boiler Footpad Lesion Score at 42 Days of Age

| | | Combined |
|---|---|---|
| TRT | TREATMENT | Avg Paw |
| NO. | (US ton) | Score |
| 1 | Negative Control | 1.03a |
| | (Basal Diets) | |
| 2 | Bacillus Blend 1X | 0.51b |
| | (0.25 lb) | |

| | |
|---|---|
| P-value; Tukey's 0.05* | P = 0.019 |

*Note:
P-value from ANOVA and means separated by Tukey's test at P = 0.05.

### Example 2

### In Vivo Studies of Efficacy of Bacillus Strains 681, A12, 101, 235, 177, 86, and 300 Against Footpad Lesions in Broiler Chickens

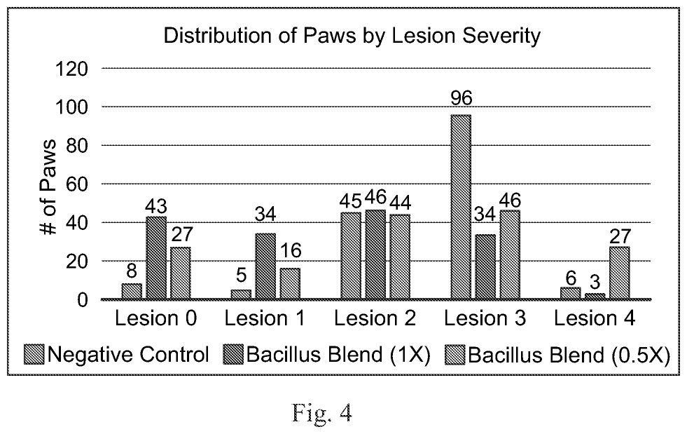

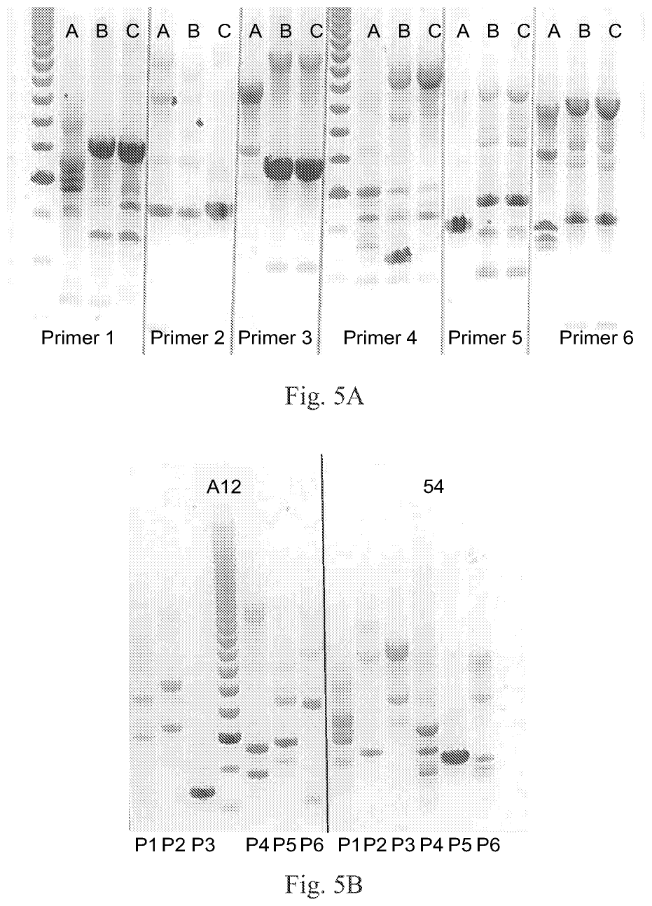

In trial 2, the efficacy of two concentrations of a Bacillus strain combination in reducing the severity of footpad lesions was determined in a floor pen study using broiler chickens exposed to used litter. This was a 42-day floor pen trial conducted with Ross 308 male chicks fed commercial type diets. Birds were fed basal diets or diets supplemented with either a combination of Bacillus strains A12 (NRRL No. B-67516), 681 (NRRL No. B-67515), 101 (NRRL No. B-67218), 235 (NRRL No. B-67219), 177 (NRRL No. B-67275), 86 (NRRL No. B-50944), and 300 (NRRL No. B-50943) at 0.25 lb/U.S. ton (1×) or the same Bacillus blend fed at ½ the concentration (0.5×). All birds were exposed to used litter beginning on day 4 and continuing through the remainder of the trial. On day 41, right and left paws were scored on 10 birds/pen (20 paws/pen) and averaged. Lesions were scored from 0 representing no lesion to 4 representing the most severe lesions. The footpad lesion score for the 1× Bacillus-treated group was significantly lower (P=0.039; FIG. 3 and Table 2) than the non-supplemented control group, with the 0.5× group intermediate. Distribution of the lesion scores provides further support for the shift toward lower severity of lesions in the Bacillus blend treatments (FIG. 4). These results provide further evidence for this Bacillus blend's effect on prevention and treatment of pododermatitis.

TABLE 2
Left, Right, and Combined Average Paw Scores on Day 41

| | | Left Paw | Right Paw | |
|---|---|---|---|---|
| TRT | TREATMENT | Score | Score | Combined Avg |
| NO. | (US ton) | (0-4) | (0-4) | Paw Score |
| 1 | Negative Control | 2.44 | 2.65 | 2.54a |
| | (Basal Diets) | | | |
| 2 | Bacillus Blend 1X | 1.46 | 1.54 | 1.50b |
| | (0.25 lb/US ton) | | | |
| 3 | Bacillus Blend 0.5X | 2.09 | 2.29 | 2.19ab |
| | (0.25 lb/US ton) | | | |

| | | | |
|---|---|---|---|
| RBC P-value; Tukey's 0.05* | 0.130 | 0.162 | 0.039 |

*Note:
P-value from ANOVA and means separated by Tukey's test at P = 0.05. Right and left paws were each scored on 10 birds/pen (20 paws/pen) and averaged. Scoring was from 0 for normal to 4 representing the most severe lesions.

### Example 3

### In Vitro Growth Inhibition of Enterococci

Enterococcus are gram-positive bacteria that commonly inhabit the gastrointestinal tracts of poultry. Transmission of Enterococcus is via the oral and/or aerosol routes as well as from skin wounds, and naturally high levels of antibiotic resistance found in E. faecalis contribute to its pathogenicity. In broiler breeders, paw lesions were correlated to systemic infection with gram-positive cocci, including Enterococcus faecalis. E. faecalis has also been specifically associated with endocarditis in broilers while other enterococci have been linked to arthritis, osteomyelitis, spondylitis, and femoral head necrosis in broiler and broiler breeder flocks. Reducing Enterococcus quantities in the gastrointestinal tract or litter is a starting point for reducing transmission. Here we demonstrate that specific Bacillus strains A12 (NRRL No. B67516), 681 (NRRL No. B67515), 101 (NRRL No. B-67218), 235 (NRRL No. B-67219), 177 (NRRL No. B-67275), 86 (NRRL No. B-50944), and 300 (NRRL No. B-50943) can inhibit the growth of Enterococcus faecalis isolates from poultry litter in vitro.

### Methods:

Enterococcus isolates were cultured from the litter environment from 21 commercial broiler houses across 5 complexes in the U.S., using BBL Enterococcosel agar (Becton Dickenson). Cultures were incubated at 37° C. for 24 hours and further propagation of presumptive positive colonies was performed in Brain Heart Infusion broth (BHI, Becton Dickenson) under the same growth conditions. Isolates were confirmed as E. faecalis via a species-specific qPCR. A total of 48 E. faecalis isolates were screened for susceptibility to culture fermentate from Bacillus strains A12 (NRRL No. B67516), 681 (NRRL No. B67515), 101 (NRRL No. B-67218), 235 (NRRL No. B-67219), 177 (NRRL No. B-67275), 86 (NRRL No. B-50944), and 300 (NRRL No. B-50943). The screening was performed in duplicate in a liquid assay, using a 1% (v/v) inoculation of a 24-hour pathogen culture into BHI broth containing >5% (v/v) of a cell-free Bacillus fermentate from each Bacillus strain. Assays were incubated for 24 hours at 37° C. under aerobic growth conditions. Optical density (OD₆₀₀) was measured, and percentage of inhibition was calculated in comparison to each pathogen isolate's positive control grown in BHI without Bacillus fermentate. Growth inhibition detected for each Enterococcus isolate and Bacillus strain combination was scored as weak (<25% inhibition, −), moderate (25-50% inhibition, +/−) or strong (>50% inhibition, +).

### Results:

The in vitro growth of litter-origin Enterococcus faecalis isolates was inhibited strongly by the presence of fermentate from one or more of Bacillus strains A12 (NRRL No. B-67516), 681 (NRRL No. B-67515), 101 (NRRL No. B-67218), 235 (NRRL No. B-67219), 177 (NRRL No. B-67275), 86 (NRRL No. B-50944), and 300 (NRRL No. B-50943). Growth inhibition of Enterococcus faecalis in the litter and footpad environment provide an example mechanism by which these specific Bacillus strains can reduce pododermatitis in poultry.

TABLE 3
In vitro inhibitory activity of specific Bacillus strains against Enterococcus faecalis
isolates from the litter environment of commercial broiler complexes. Growth
inhibition is classified as high (+), moderate (+/−) or low (−).

| Enterococcus faecalis | | Bacillus | Bacillus | Bacillus | Bacillus | Bacillus | Bacillus |
|---|---|---|---|---|---|---|---|
| Isolate | Origin | 86 | 177 | 235 | 300 | A12 | 681 |
| Path1 | Complex1 Farm1 | +/− | − | +/− | + | + | + |
| Path2 | Complex1 Farm2 | + | − | + | + | − | + |
| Path3 | Complex1 Farm3 | + | − | + | + | + | + |
| Path4 | Complex2 Farm1 | + | − | − | + | + | + |
| Path5 | Complex2 Farm2 | + | − | + | + | + | + |
| Path6 | Complex2 Farm3 | + | − | +/− | + | + | + |
| Path7 | Complex1 Farm4 | + | − | + | + | + | + |
| Path8 | Complex1 Farm1 | +/− | − | + | + | + | + |
| Path9 | Complex3 Farm1 | − | − | + | + | + | +/− |
| Path10 | Complex1 Farm3 | − | − | + | + | + | +/− |
| Path11 | Complex2 Farm1 | − | − | + | + | + | + |
| Path12 | Complex3 Farm2 | +/− | − | + | + | + | + |
| Path13 | Complex2 Farm3 | − | − | + | + | +/− | − |
| Path14 | Complex3 Farm2 | +/− | − | +/− | + | + | + |
| Path15 | Complex1 Farm4 | + | − | + | + | + | + |
| Path16 | Complex1 Farm1 | +/− | − | + | + | + | + |
| Path17 | Complex 3 Farm1 | + | − | + | +/− | + | + |
| Path18 | Complex1 Farm3 | +/− | − | + | + | + | + |
| Path19 | Complex2 Farm1 | +/− | − | + | + | + | + |
| Path20 | Complex3 Farm2 | +/− | +/− | +/− | + | + | +/− |
| Path21 | Complex2 Farm3 | +/− | − | + | + | + | +/− |
| Path22 | Complex3 Farm2 | +/− | − | + | + | +/− | + |
| Path23 | Complex1 Farm1 | + | − | + | + | + | + |
| Path24 | Complex3 Farm1 | + | − | + | + | + | + |
| Path25 | Complex1 Farm3 | + | − | + | + | + | + |
| Path26 | Complex2 Farm1 | +/− | − | + | + | + | +/− |
| Path27 | Complex3 Farm2 | − | − | + | + | + | +/− |
| Path28 | Complex2 Farm3 | + | − | + | + | + | + |
| Path29 | Complex 4 Farm 1 | +/− | − | − | − | + | − |
| Path30 | Complex 4 Farm 1 | +/− | − | + | + | + | + |
| Path31 | Complex 4 Farm 1 | − | − | − | + | + | + |
| Path32 | Complex 4 Farm 1 | − | − | + | + | + | +/− |
| Path33 | Complex 4 Farm 1 | +/− | − | + | + | + | + |
| Path34 | Complex 4 Farm 1 | + | +/− | − | − | + | +/− |
| Path35 | Complex 4 Farm 2 | +/− | − | + | + | +/− | +/− |
| Path36 | Complex 4 Farm 2 | +/− | − | + | + | +/− | +/− |
| Path37 | Complex 4 Farm 2 | + | − | + | + | + | +/− |
| Path38 | Complex 4 Farm 2 | +/− | − | + | + | + | + |
| Path39 | Complex 4 Farm 2 | − | − | + | + | + | +/− |
| Path40 | Complex 4 Farm 2 | − | − | + | + | +/− | − |
| Path41 | Complex 4 Farm 2 | +/− | − | + | + | + | + |
| Path42 | Complex 4 Farm 3 | +/− | − | + | + | + | +/− |
| Path43 | Complex 4 Farm 3 | +/− | − | + | + | + | + |
| Path44 | Complex 5 Farm 1 | − | − | +/− | + | + | +/− |
| Path45 | Complex 5 Farm 2 | +/− | − | − | + | + | + |
| Path46 | Complex 5 Farm 2 | +/− | − | +/− | + | + | +/− |
| Path47 | Complex 5 Farm 2 | − | − | + | + | + | + |
| Path48 | Complex 5 Farm 2 | − | − | + | + | + | + |

### Example 4

### Strain Identification and Uniqueness

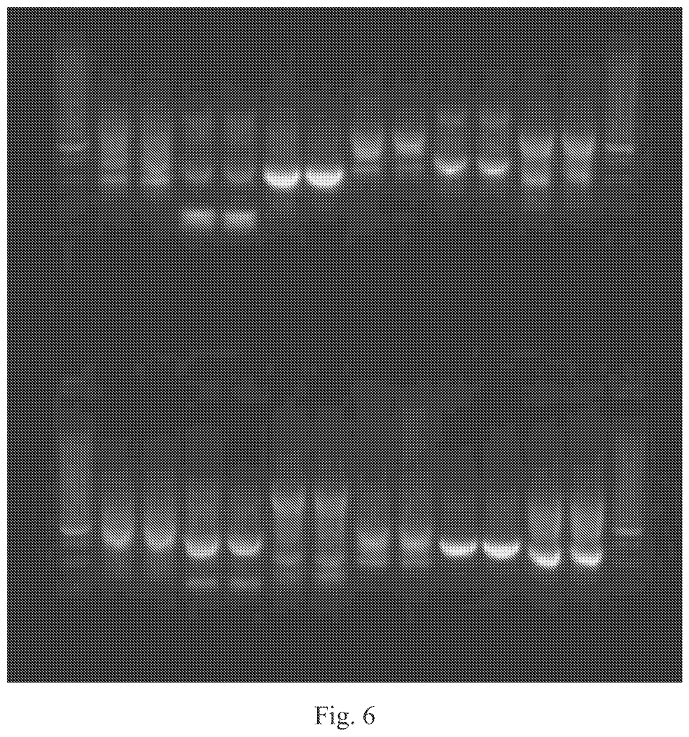

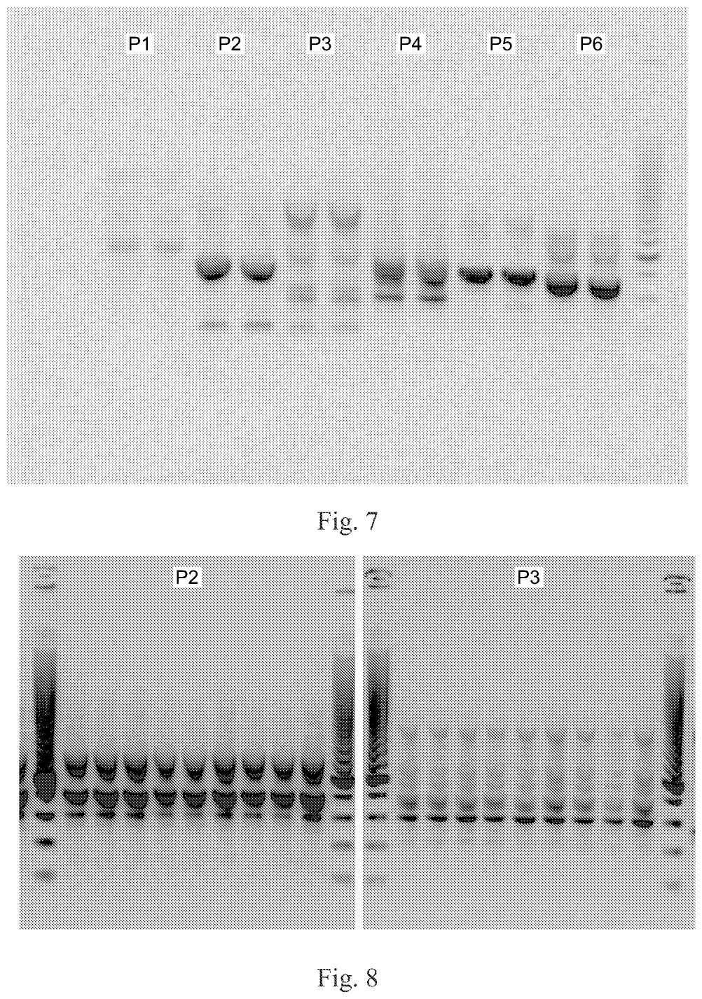

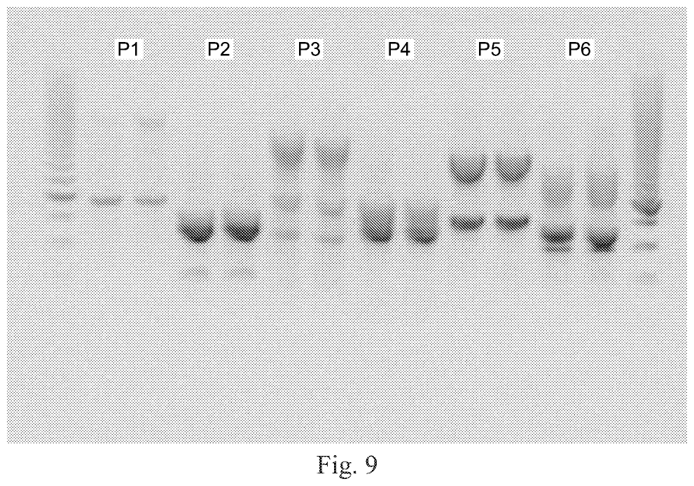

The Randomly Amplified Polymorphic DNA PCR method (hereafter referred to as RAPD-PCR) was used to identify genetic variability of each strain. Preparation of the DNA to be used in the RAPD-PCR reaction was done by using the QIAGEN® Tissue and Blood single column kit (QIAGEN®, Venlo, The Netherlands). To obtain DNA, an overnight culture was prepared, struck for purity, pelleted, and DNA was extracted following the manufacturer's protocol. The RAPD-PCR reaction was performed with the following run conditions in a thermal cycler: 95° C. 5 min, followed by 45 cycles of (95° C. 1 min, 36° C. 1 min, 72° C. 2 min), followed by 72° C. 7 min, and finished with a 4° C. indefinite hold to preserve the product. The RAPD-PCR product was analyzed by gel electrophoresis. FIGS. 5A and 5B and 6 to 9 illustrate RAPD-PCR results for Bacillus strains A12, 681, 101, 235, 177, 86, and 300.

1-4. (canceled)

5. A method of controlling a detrimental effect of Enterococcus in an animal, the method comprising the steps of administering to an animal a feed composition or drinking water comprising an effective amount of an additive comprising an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

6. The method of claim 5 wherein controlling the detrimental effect of Enterococcus comprises reducing or preventing Enterococcus disease in the animal.

7. The method of claim 5 wherein controlling the detrimental effect of Enterococcus comprises reducing Enterococcus disease inhibiting Enterococcus in the animal.

8. The method of claim 5 wherein the strains administered comprise Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), and 177 (NRRL No. B-67275).

9. The method of claim 5, wherein the Bacillus strain prevents or reduces pododermatitis in the animal.

10-12. (canceled)

13. A commercial package for inhibiting Enterococcus and/or preventing or reducing pododermatitis in an animal wherein the commercial package comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

14. A feed additive for an animal feed, an animal feed composition, or an additive for drinking water of an animal, for inhibiting Enterococcus and/or preventing or reducing pododermatitis in an animal wherein the additive comprises an isolated Bacillus strain selected from the group consisting of Bacillus strain A12 (NRRL No. B-67516), 681 (NRRL No. B-67515), 300 (NRRL No. B-50943), 101 (NRRL No. B-67218), 235 (NRRL No. B-67219), 86 (NRRL No. B-50944), 177 (NRRL No. B-67275), a strain having all of the identifying characteristics of Bacillus strain A12 (NRRL No. B67516), a strain having all of the identifying characteristics of Bacillus strain 681 (NRRL No. B67515), a strain having all of the identifying characteristics of Bacillus strain 300 (NRRL No. B-50943), a strain having all of the identifying characteristics of Bacillus strain 101 (NRRL No. B-67218), a strain having all of the identifying characteristics of Bacillus strain 235 (NRRL No. B-67219), a strain having all of the identifying characteristics of Bacillus strain 86 (NRRL No. B-50944), a strain having all of the identifying characteristics of Bacillus strain 177 (NRRL No. B-67275), and combinations thereof.

15. (canceled)

16. (canceled)

17. The feed additive, feed composition, or additive for the drinking water of the animal of claim 14 in the form of a concentrate.

18. The feed additive, feed composition, or additive for the drinking water of the animal of claim 14 in dry form.

19. The feed additive, feed composition, or additive for the drinking water of the animal of claim 14 in pelleted form.

20. The commercial package of claim 13 wherein the strains are in a form selected from the group consisting of a powder, a liquid, and a pellet form.

21. The commercial package of claim 13 further comprising a carrier for the Bacillus strains.

22. The commercial package of claim 21 wherein the carrier is selected from the group consisting of a bran, rice hulls, a salt, a dextrin, and combinations thereof.

23. The commercial package of claim 13 in a bag, optionally a plastic bag.

24. (canceled)

25. The method of claim 5 wherein said additive is in powder form.

26. The method of claim 5 wherein said additive is in liquid form.

27. The commercial package of claim 13 in a container for commercial use.

28. The commercial package of claim 13 further comprising a binder.

29. The commercial package of claim 28 wherein the binder is selected from the group consisting of clay, yeast cell wall components, aluminum silicate, and glucan, or combinations thereof.

30-31. (canceled)
